(19) United States
(12) Patent Application Publication
(10) Pub. No.: US 2014/0358167 A1
(43) Pub. Date: Dec. 4, 2014

(54) ANASTOMOTIC SLEEVE DEVICE

(71) Applicant: David N. Armstrong, Atlanta, GA (US)

(72) Inventor: David N. Armstrong, Atlanta, GA (US)

(21) Appl. No.: 14/289,266

(22) Filed: May 28, 2014

Related U.S. Application Data
(60) Provisional application No. 61/828,272, filed on May 29, 2013.

(51) Int. Cl. A61B 17/115; A61B 17/068

(52) U.S. Cl. CPC A61B 17/115; A61B 17/068
USPC 606/153; 227/175.1

(57) ABSTRACT

An anastomotic sleeve or “cup” protection device incorporating distal and proximal sleeves or “cups”, also incorporating a staple-line buttress. The proximal colon is inserted into the proximal sleeve or cup and the shaft of the stapler anvil is passed through a hole in the closed end of the sleeve or cup. The distal sleeve or cup is placed over the head of the staple shaft, and inserted into the rectum for anastomosis. Closure and firing of the stapler creates a staple buttress line at the anastomosis, the proximal sleeve prevents leakage from the proximal colon and the distal sleeve protects the distal colon or rectum from anastomotic leak.

### CROSS-REFERENCE TO RELATED APPLICATIONS

This application claims priority to provisional patent application U.S. 61/828,272 filed May 29, 2013, which is hereby incorporated by reference herein.

### FIELD OF THE INVENTION

The Technical Field relates to medical devices for the prevention of leakage associated with anastomotic colon resection.

### BACKGROUND

Anastomotic leak after colon resection such as sigmoid colectomy or low anterior resection occurs in 2-20% of cases. This results in leakage of bowel content into the peritoneal cavity with resulting peritonitis and potentially death.

Anastomotic leak can occur for several reasons: Tension on the anastomosis, inadequate blood supply to the anastomosis, or disruption of the staple line are the most common reasons. Leaks typically occur within a centimeter of two of the anastomosis on either the proximal or distal side of the staple line. Anastomotic leaks allow stool to flow out of the bowel and into the peritoneal or pelvic cavity causing peritonitis and even death.

### SUMMARY

In a first aspect, the invention pertains to a system for performing a medical procedure comprising a stapler, and a support structure. The stapler generally comprises a detachable anvil head comprising an anvil surface and a hollow rod comprising a lumen protruding from the anvil surface, a stapler shaft comprising a stapler surface and a spike protruding from the stapler surface, and a shape cutter. The anvil surface can comprise a groove. The stapler surface generally comprises a staple port and a cutter port. The cutter can advance through the cutter port, and the spike is proportioned to fit inside the lumen of the hollow rod. The support structure can comprise a first shield piece comprising a first support surface and at least one first wall comprising a first bottom edge wherein the first bottom edge is attached to the first support surface and a second shield piece comprising a second support surface. The second shield piece generally is not attached to the first shield piece. The first support surface and/or the second support surface can comprise a hole, and the first support surface and the second support surface can align with the anvil surface and the stapler surface to provide for the delivery of a staple through the first support surface and the second support surface.

In another aspect, the invention pertains to a method for forming an anastomosis generally comprising placing a first attachment site near the distal end of a colon segment into contact with a first shield piece comprising a first surface, placing a second attachment site of the rectum into contact with a second shield piece comprising a second surface, inserting a tip of a pointed attachment member through the first surface, tissue at the first attachment site, the second surface, and tissue at the second attachment site, creating an affixed section, and cutting through the affixed section to create a lumen. The first shield piece can comprise at least one first wall comprising a first bottom edge wherein the first bottom edge is attached to the first surface and/or the second shield piece can comprise at least one second wall comprising a second bottom edge wherein the second bottom edge is attached to the second surface.

In a further aspect the invention pertains to a medical apparatus for protecting an anastomosis comprising a first shield piece comprising a first surface and a second shield piece comprising a second surface. The second shield piece generally is not attached to the first shield piece. The first shield piece comprises at least one first wall comprising a first bottom edge wherein the first bottom edge is attached to the first surface and/or the second shield piece comprises at least one second wall comprising a second bottom edge wherein the second bottom edge is attached to the second surface. The first surface and/or the second surface comprises a hole, and the first shield piece and the second shield piece each independently comprise a sheet of biocompatible polymer and/or collagenous material with an average thickness of no more than about 5 millimeters and are sterile.

### BRIEF DESCRIPTION OF DRAWINGS

FIG. 1 is a perspective view of a two cup embodiment.

FIG. 2 is a perspective view of an anastomosis stapler.

FIG. 3 is a perspective view of a stapler reinforcement mechanism or buttress.

FIG. 4 is a perspective view of a two cup support structure where the one cup is depicted in use prior to anastomosis.

FIG. 5 is a perspective view of a two cup support structure where the second cup is depicted in use prior to anastomosis.

FIG. 6 is a perspective view of a two cup support structure in use prior to anastomosis.

FIG. 7 is a perspective view of a two cup support structure in use during to anastomosis.

FIG. 8 is a perspective view of a two cup support structure in use during to anastomosis, when the stapler has been advanced to the staple line.

FIG. 9 is a sectional view of a two cup supports structure in use after anastomosis with the cross section taken longitudinally through the approximate center of the colon.

FIG. 10 is a perspective view of one possible alternative embodiment of a support structure.

FIG. 11 is a perspective view of another alternative embodiment of a support structure.

FIG. 12 is a perspective view of a further alternative embodiment of a support structure.

FIG. 13 is a sectional view of a support structure with a third disk during anastomosis with the cross section taken longitudinally through the approximate center of the colon.

FIG. 14 is a perspective view of a two cup support structure in use after anastomosis where the proximal cup is located within the colon and the distal cup is located within the rectum.

FIG. 15 is a perspective view of a two cup support structure in use after anastomosis with the proximal cup located outside the colon and the distal cup located outside the rectum.

FIG. 16 is a perspective view of a two cup support structure in use after anastomosis with the proximal cup located inside the colon and the distal cup located outside the rectum.

### DETAILED DESCRIPTION

Improved support structures are described to more effective reinforce staple lines or similar fastener lines introduced during an anastomosis procedure. In some embodiments, the improved device comprises two sleeves, e.g., two cylinders or cups of absorbable or bio-remodelable material located around the proximal and within the distal aspect of an anastomosis. Each sleeve incorporates a peripheral sleeve, a closed end, and a second open end, in the form of a cup. Suitable alternative embodiments of the device are described further below.

After resecting a portion of left colon, sigmoid colon or rectum for cancer or diverticular disease, there remains two ends of bowel to be joined or anastomosed: a proximal end usually somewhere in the left colon, and a stapled-off distal end usually located in the rectum, depending on the level of resection.

To perform an end-to-end anastomosis, an End-to-End (EEA) stapler is used comprising a detachable anvil head and a stapler shaft. The anvil is sutured in the proximal bowel, and the hollow shaft protrudes. The shaft of the stapler is inserted into the rectum, an advancable spike is advanced through the sutured proximal rectum, and is attached to the hollow anvil shaft. The anvil and the stapler shaft are then approximated and fired, wherein two rows of circumferential staples are deployed around the periphery of the two ends of bowel, anastomosing them together. Simultaneously a circular knife creates a lumen within the staple line creating an anastomosis.

While fastening with staples to perform the EEA is the focus of the discussion herein, other fastening methods can be combined with the staple methods or otherwise adapted to take advantage of the supports structures for forming an anastomosis, with other methods including, for example, suturing, applying surgical adhesives or combinations thereof. Similarly, other methods for forming a lumen through the implanted support structure are contemplated including, for example, scalpel incisions.

Stapler reinforcement devices or buttresses reinforce the staple line itself, distribute tension around the anastomosis, increase burst pressure and make a mechanically stronger anastomosis. However, the reinforcement devices described herein are better able to protect the few centimeters proximal and distal to the staple line, and prevent leakage.

Coloshield is an endoluminal tube inserted via a separate incision in the colon, and straddles the anastomosis and prevents leakage. The device is biodegradable and is passed after several days. The C-shield device is a condom like device attached to the anvil head of an EEA stapler. After firing the stapler, the sleeve deploys around the distal anastomosis protecting the distal bowel from leakage, but the bowel proximal to the anastomosis is left unprotected. Intraluminal devices have not achieved widespread use as summarized in an article “Can intraluminal devices prevent or reduce colorectal anastomic leakage: A review,” Morks et al., World J. Gastroenterology, Oct. 28, 2011, 17(40): 4461-4469. The present devices provide the advantages of relative simplicity of use and involving a modest amount of time, yet providing an effective inhibitor of anastomic leakage.

As described herein, an improved alternative to these products incorporates an apparatus to reinforce EEA staple lines between two segments of bowel and to prevent anastomotic leakage from the vicinity of the anastomosis. An embodiment of the apparatus comprises two cups of similar form or shape to two disposable medicine cups, deployed with the bases of the cups opposing each other. One of the cups is placed over the distal end of the proximal colon segment, with the hollow shaft of the stapler anvil protruding through a hole at the base of the cup. The second cup is placed over the EEA staler shaft as it is inserted into the rectum, and advanced up to the staple line. Suitable alternative embodiments of the apparatus are described below for incorporation into the system.

The spike of the stapler shaft is advanced through the stapled-off rectum and attached to the anvil. The stapler is then closed and fired. After firing the stapler, the base of the cups forms a staple line buttress, and the sides of the cups prevent anastomotic leak by encircling the periphery of the proximal colon, and the inner aspect of the rectum. Simultaneously a circular knife in the stapler creates a lumen within the anastomosis.

The apparatus prevents anastomotic leak from the vicinity of the anastomosis and incorporates a staple line buttress or reinforcement. The cups or sleeves may be both located on the inner aspect of the bowel, both on the outer aspect of the bowel, or any combination thereof. The material the apparatus is made out of may be absorbable, semi absorbable non absorbable or bioremodelable. It may be synthetic or biological.

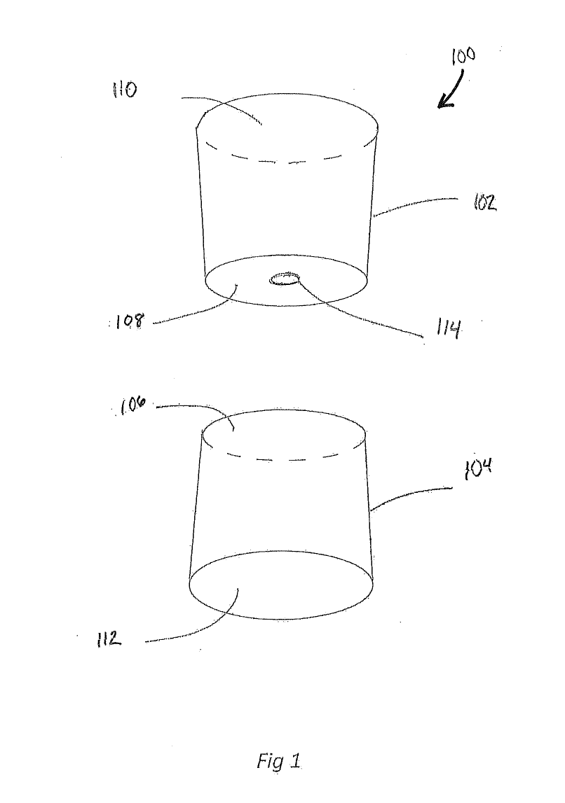

FIG. 1 depicts an embodiment of a device to reinforce EEA staple lines between two segments of bowel, and to prevent anastomotic leakage from the vicinity of the anastomosis. Device 100 consists of two cups 102, 104 of similar form or shape to two disposable medicine cups, deployed with bases 106, 108 opposing each other and openings 110, 112 facing away from each other. The cups 102, 104 may be generally cylindrical or conical or a hybrid of both or other suitable shape, although suitable more deformable shapes may have adjustable shapes.

In use, cup 102 is placed over the distal end of the proximal colon segment, with the hollow shaft of the stapler anvil protruding through hole 114 in base 108. Cup 104 is placed over the EEA stapler head, which is inserted into the rectum, and advanced to the staple line. The spike of the stapler shaft is advanced and attached to the anvil, and then the stapler is closed and fired. After firing the stapler, a lumen is created between the bowel and bases 106, 108 form a staple line reinforcer or buttress. The sides of the cup prevent anastomotic leak by encircling the periphery of the proximal colon, and the inner aspect of the rectum.

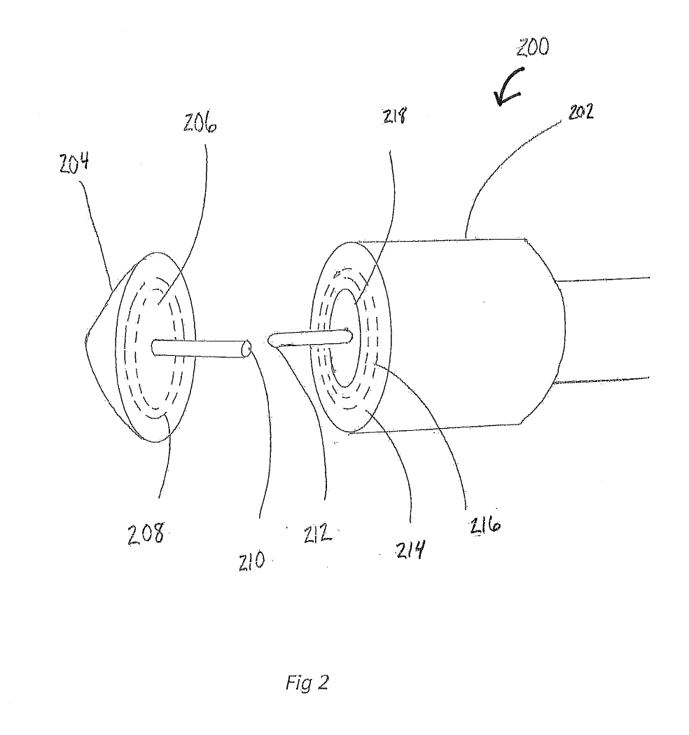

FIG. 2 depicts end-to-end anastomotic (EEA) staple gun 200 comprising stapler shaft 202 and detachable anvil 204. Detachable anvil 204 comprising flat anvil surface 206 containing grooved contours 208 which bend the staples into a “D” or “B” form, so joining or anastomosing the bowel. Anvil 204 also incorporates hollow shaft 210 to accept advancable stapler spike 212. Stapler shaft 202 incorporates stapler head 214 which contains two concentric circles of staples 216 and inner circular blade 218 to cut the bowel and form a lumen. Stapler head 214 also contains spike 212 which is advanced and attached to anvil 204.

In use, the anvil is sutured into the proximal segment of colon prior to anastomosis. The shaft of the stapler is inserted into the rectum and advanced to the staple line. The spike is advanced through the base of the distal cup and the stapled-off rectum and connected to the hollow shaft of the anvil. The stapler is then securely closed, wherein the head and anvil are opposed and the stapler is fired. The bowel is anastomosed by two concentric rows of staples, and simultaneously or subsequently a circular blade creates the anastomotic lumen.

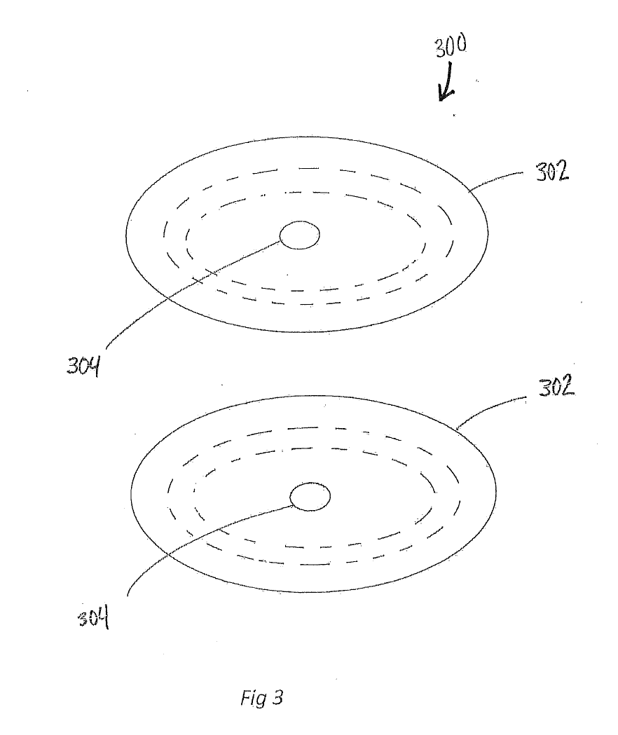

FIG. 3 depicts staple buttresses 300 incorporate disks 302 of material with holes 304 which are placed on the proximal anvil and the head of the stapler. In use, the anvil shaft and stapler spike are passed through holes 304 in the staple line reinforcers. Firing the staples incorporates both ends of the bowel, as well as both buttresses 300 which mechanically reinforces the anastomosis.

These stapler reinforcement devices or buttresses reinforce the staple line itself, distribute tension around the anastomosis, increase burst pressure and make a mechanically stronger anastomosis. The problem with the staple line buttresses is that anastomotic leaks typically extend a few centimeters proximal or distal from the anastomosis, for instance due to an inadequate blood supply or tension on the anastomosis. However no staple line reinforcement devices protect the bowel proximal or distal to the anastomosis, so leakage of bowel content can still occur.

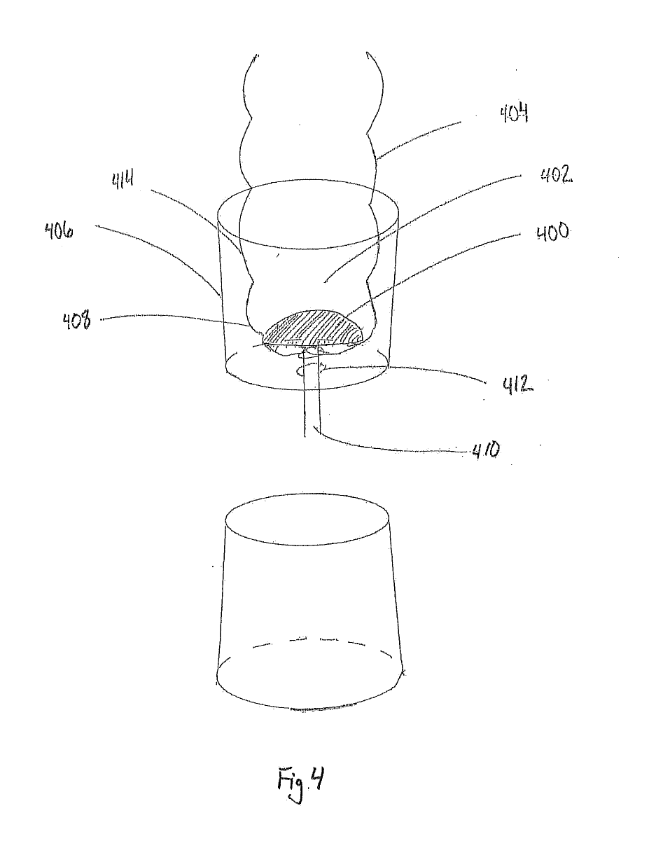

In FIG. 4 anvil 400 is sutured into the lumen 402 of the proximal colon 404 as in conventional stapling. The proximal cup 406 is inserted over end of colon 408 to be anastomosed. Central hollow shaft 410 of anvil 400 is passed through hole 412 in the closed end of cup 406. The proximal cup 406 is therefore located around the periphery 414 or outer aspect of the proximal colon.

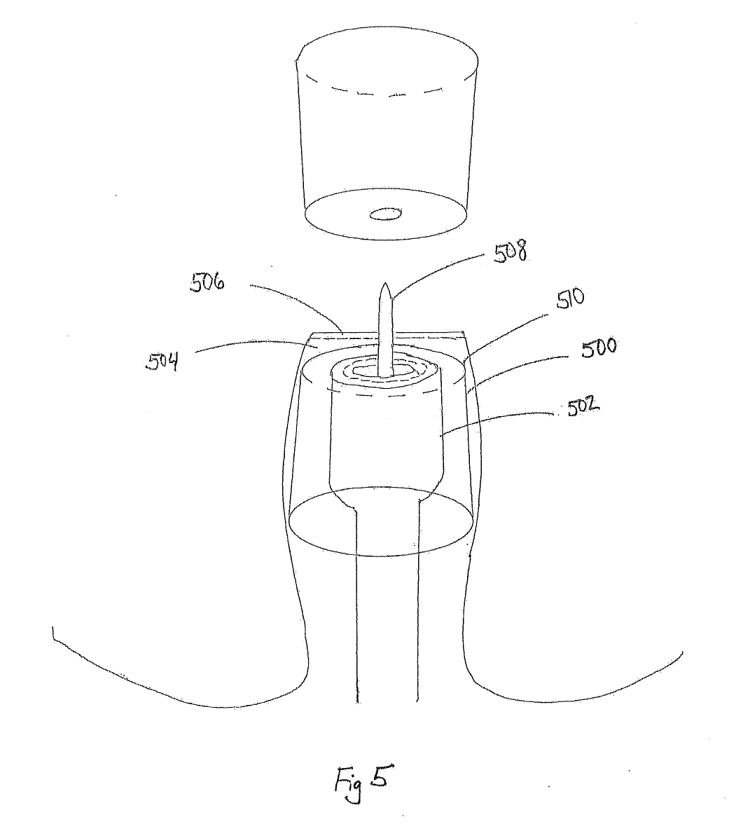

In FIG. 5 the distal aspect of sleeve 500 is placed over the end of stapler shaft 502. The stapler shaft is inserted inside rectum 504 and advanced to staple line of 506 transected rectum as in a conventional stapler. Spike 508 is advanced through the closed end 510 of the sleeve 500, which becomes a staple line bolster.

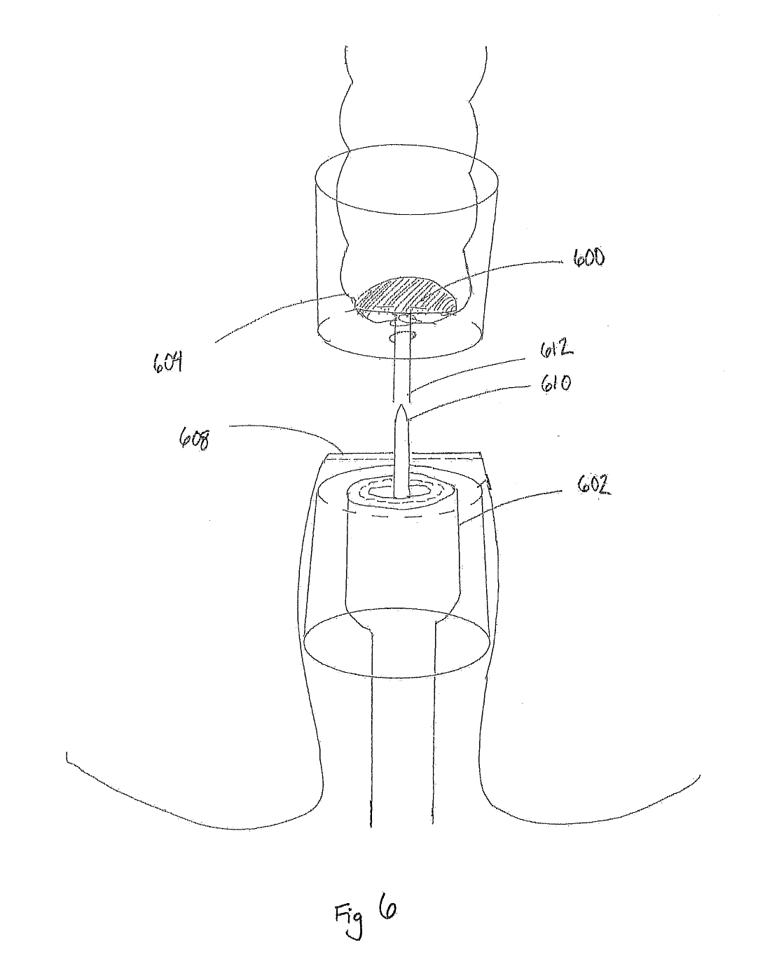

In FIG. 6 anvil 600 is closed onto stapler head 602 prior to anastomosing colon 604 to stapled-off rectum 608. Spike 610 is inserted into hollow shaft 612 of the anvil 600.

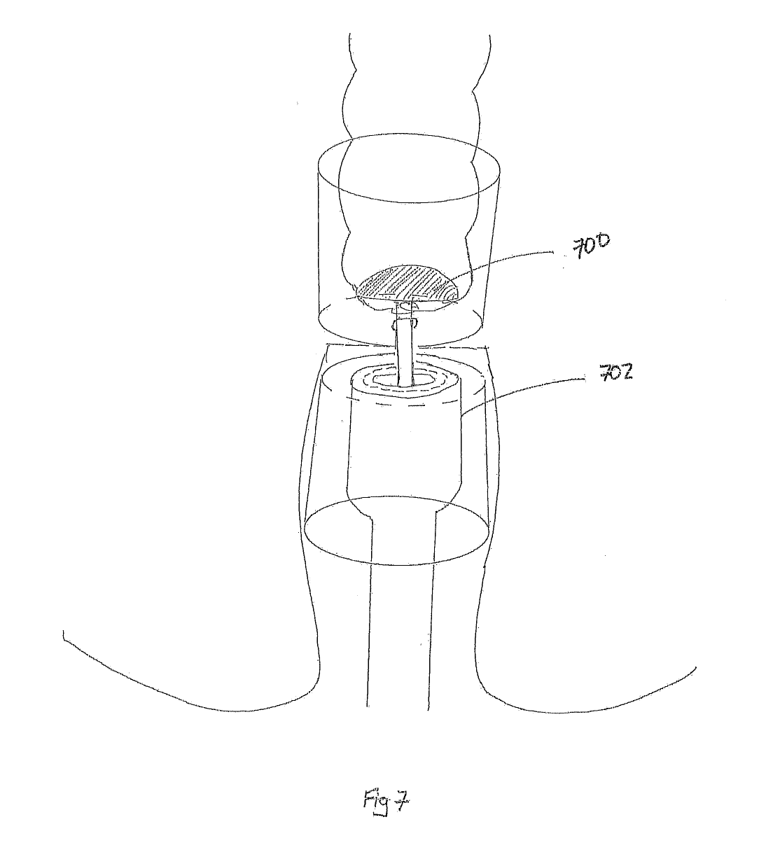

In FIG. 7 anvil 700 closed onto head 702 of the stapler.

In FIG. 8 anvil 800 is closed onto stapler head 802 and the stapler is fired. Two circumferential rows of staples are deployed through bowel wall 804, and through the two closed ends of sleeve 806, 808, forming reinforcement 810. Simultaneously or shortly thereafter, a circular blade forms lumen 812 between the two ends of the bowel.

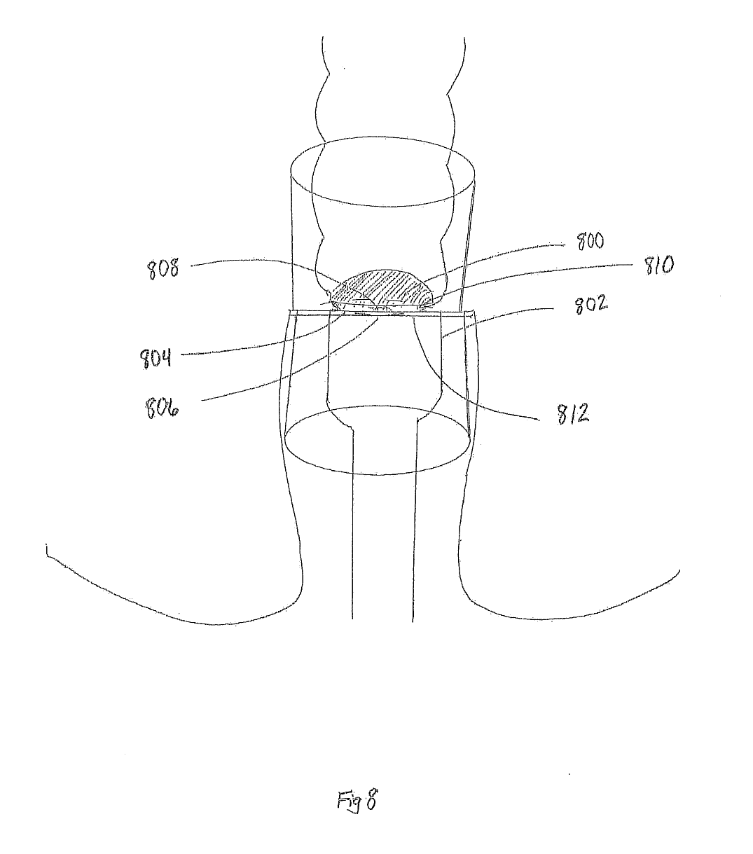

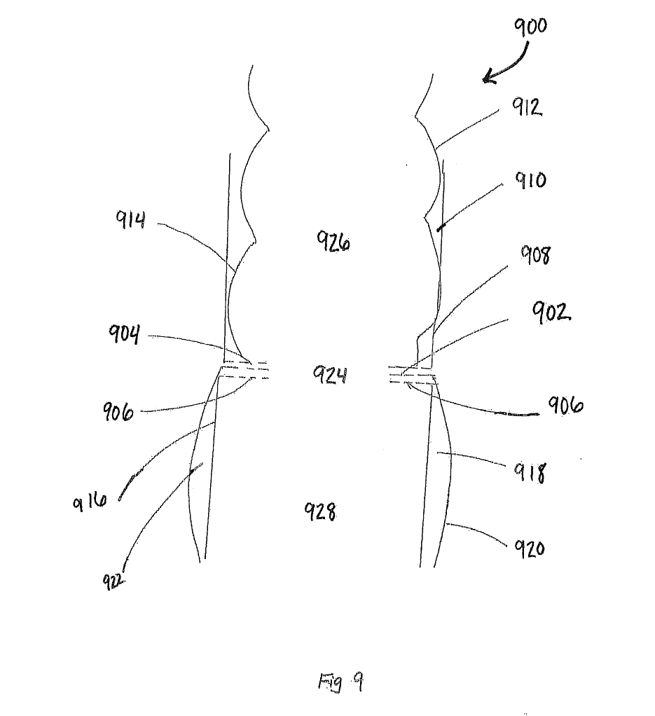

FIG. 9 illustrates a cross section of staple anastomosis 900. The staple line buttress 902, 904 reinforces anastomosis 906. Proximal sleeve 908 located around periphery 910 of proximal colon 912 protects proximal aspect 914 of the anastomosis. Distal sleeve 916 located in inner aspect 918 of rectum 920 reinforces distal aspect 922 of the anastomosis. Anastomotic lumen 924 forms a channel between proximal colon 926 and rectum 928.

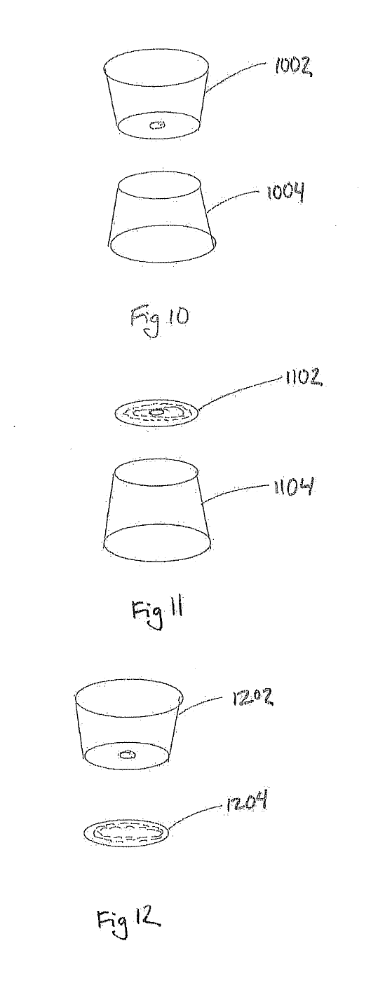

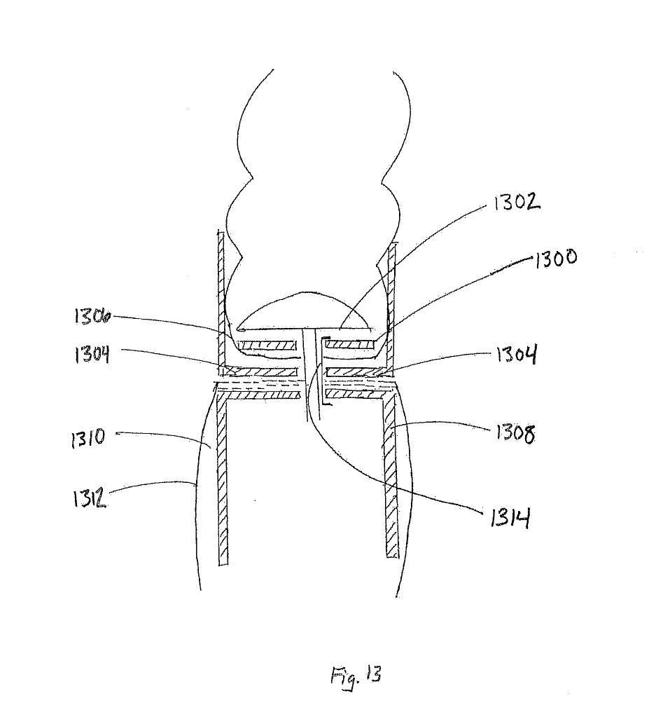

FIGS. 10-12 illustrate various possible additional or alternative embodiments of the device. For example, the device may incorporate two “mirror-image” sleeves or cups 1002, 1004 as shown in FIG. 10. The device may comprise simple proximal disk 1102 located on the flat surface of the stapler anvil and distal sleeve 1104 or cup as shown in FIG. 11. The device may comprise proximal sleeve 1202 and distal disk 1204 located on the surface of the stapler head as shown in FIG. 12. In FIGS. 11 and 12 the disk may be located directly on the surface of the anvil surface as in staple line reinforcement described previously or between the opposing bowel surfaces.

In FIG. 13 third disk 1300 is provided on the surface of anvil head 1302. Proximal sleeve 1304 is deployed around the circumference of proximal colon 1306 and distal sleeve 1308 is deployed within lumen 1310 of distal bowel 1312 or rectum. All three buttresses are joined together with intervening bowel wall 1314 like a “club sandwich”. The three buttresses provide a more secure and integral device to prevent anastomotic leak or separation of the proximal and distal portions of the anastomosis.

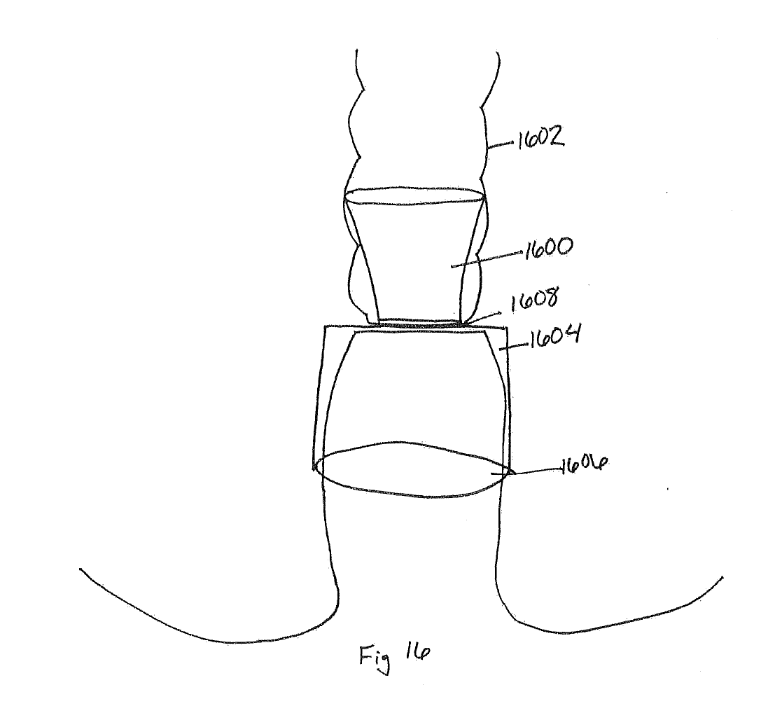

Herein, distal and proximal are described relative to the body of the patient. When incorporated into an anastomosis the sleeves, cups, and disks are layered with the tissue to be incorporated into anastomosis in various configurations in order to provide the protection desired. Both the distal and proximal sleeves and/or disks may be located on the outer aspect of the proximal bowel and the rectum or distal bowel and anastomosis. In FIG. 15 proximal sleeve 1500 is located outside of colon 1502. Distal sleeve 1504 is located outside rectum 1506. Proximal sleeve 1500 is anastomosed to distal sleeve 1504 along staple line 1508. Both the proximal and distal sleeves and/or disks may be located on the inner aspect of the proximal bowel and the rectum or distal bowel and anastomosis. In some embodiments, the proximal sleeve or disk is on the inner aspect of the colon and the distal sleeve or disk located on the outer aspect of the rectum or distal colon. In FIG. 16 proximal sleeve 1600 is located inside of colon 1602. Distal sleeve 1604 is located outside rectum 1606. Proximal sleeve 1600 is anastomosed to distal sleeve 1604 along staple line 1608. In embodiments where placing protection outside the rectum or distal colon is desired, the sleeve or disk may be placed over the distal aspect of the rectum or colon manually by the surgeon.

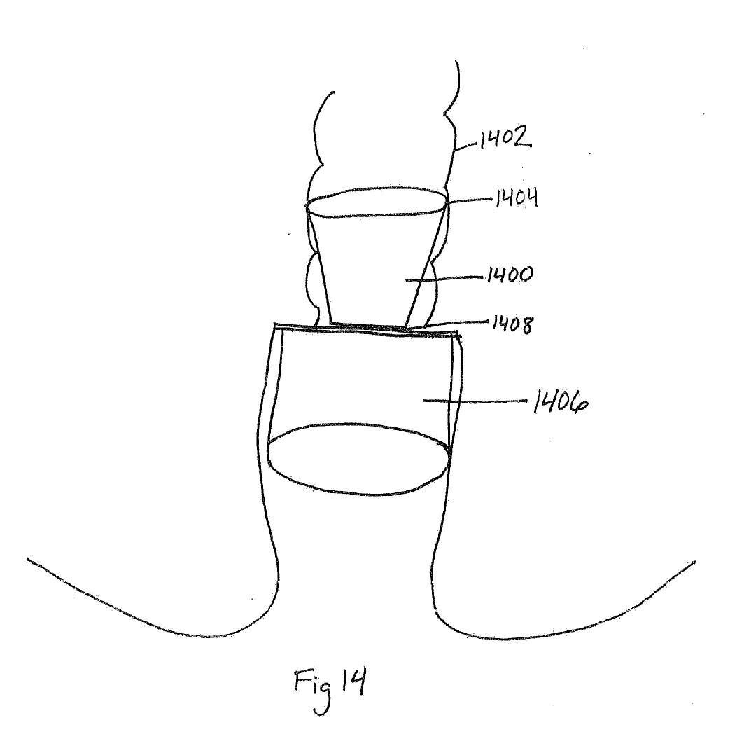

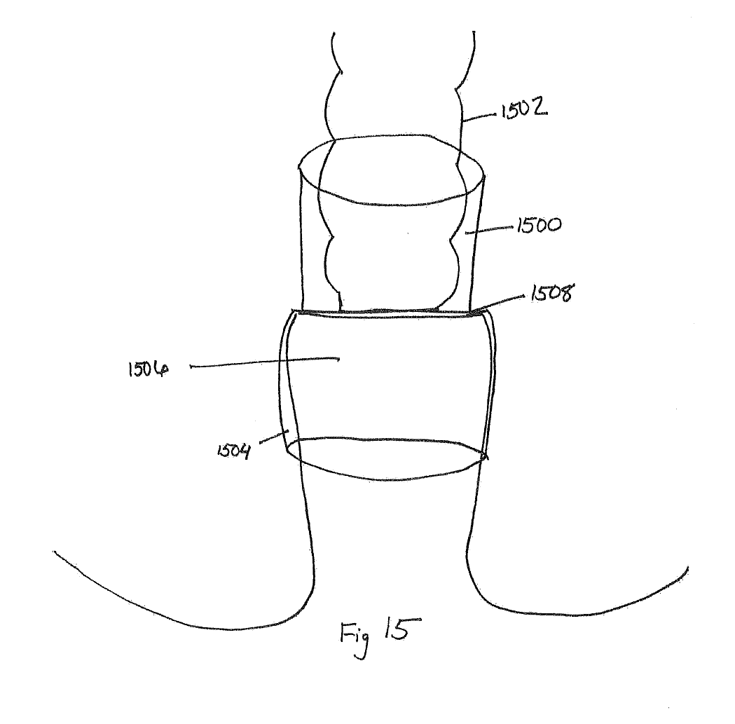

In yet a further embodiment, the proximal sleeve may be held vertical by a radially extendable ring at the proximal circumference of the proximal cup or sleeve. The ring keeps the sleeve in situ by lateral pressure against the wall of the colon. As shown in FIG. 14, proximal sleeve 1400 is placed within the lumen of colon 1402. Radially extendable ring 1404 is in contact with the wall of colon 1402. Distal sleeve 1406 is located on the outer aspect of the rectum. Proximal sleeve 1400 is anastomosed to distal sleeve 1406 along staple line 1408.

In a further embodiment, a proximal sleeve or cuff is located on the inner aspect of the proximal colon and is secured in place by a semi-rigid, e.g., radial extendable, framework, such as that incorporated into current colon stent designs. Biocompatible radially extendable stent frames are established for placement in various patient lumens. The framework may be affixed or attached on the inner aspect of the sleeve or be an integral part of the sleeve with the framework coated on both sides by a suitable material as describe herein. Similarly, in further embodiments the distal sleeve or cuff is located on the inner aspect of the rectum and is secured in place by a semi-rigid framework, such as that incorporated into current colon stent designs. The framework may be affixed or attached on the inner aspect of the sleeve or be an integral part of the sleeve with the framework coated on both sides by a suitable material as describe herein. Additionally, both proximal and distal sleeves may be held in place by the framework as described above.

The framework may be made of Nitinol, which holds the sleeve in place and prevents prolapse of the sleeve through the anastomosis. The Nitinol, a shape memory alloy, can expand or change shape once deployed in the body, and can assume a form or shape to secure the device or devices in place. The extendable frame may be located in the sleeves of the device and generally does not extend into the base or bases, so avoiding incorporation into the staple line buttresses, and preventing anastomotic disruption at anastomosis.

In further embodiments, both or either outer sleeves may incorporate a semi-rigid or extendable framework to provide stability and maintain the sleeves in place.

### Shape and Dimensions

The device may be any shape suitable to both buttress the anastomosis and protect the site proximal and/or distal to the anastomosis. In some embodiments a device comprises a disk of any suitable shape including, for example, circle, oval, polygons with rounded corners or the like. The disk may be substantially flat, may be concave or convex, or may comprise recesses. The proximal and/or the distal element may further comprise a wall attached to the disk at least partially surrounding the parameter of the disk. The walls may be substantially perpendicular to the disk, or at an angle of no more than about 120° relative to the disk, no more than about 110° relative to the disk, or no more than about 100° relative to the disk. The walls may also lean toward the center of the disk at an angle of no more than about 60° relative to the disk, no more than about 70° relative to the disk, or no more than about 80° relative to the disk. People of ordinary skill in the art will immediately appreciate that all values and ranges within the expressly stated ranges are contemplated, and are within the present disclosure. To the extent that the material of the wall is somewhat flexible, the structure can be expanded with light pressure, such as with gentle finger expansion, to evaluate the shape.

In some embodiments, the proximal element of the device comprises a hole in the disk to allow for the hollow rod of the anvil shaft. The hole may be sized to accommodate the hollow rod. It may be slightly larger than the hollow rod or the same size as the hollow rod. In further embodiments the distal element of the device also comprises a hole.

The diameter of the device can be selected based on the diameter of the end-to-end anastomotic stapler used. Conventional sizes are 25 mm, 28 mm, 29 mm, 31 mm or 33 mm. Conventional staplers include devises manufactured by Ethicon, Covidien or Intuit staplers. The length of the sleeve or wall of the current device may be about 1 mm to about 10 cm, in additional embodiments from about 2.5 mm to about 7.5 cm, in some embodiments from about 1 cm to about 5 cm, and in further embodiments from about 2 cm to about 4 cm long. The thickness of the material may be about 10 microns to about 1 mm and in further embodiments from about 100 microns to about 0.5 mm thickness depending on the material. A person of ordinary skill in the art will recognize that additional ranges are contemplated and are within the present disclosure. Persons of ordinary skill in the art will immediately appreciate that all values and ranges within the expressly stated ranges are contemplated.

### Materials

The material of the device may be non-absorbable, semi-absorbable, absorbable or bioremodelable. As used herein, absorbable materials refer to materials in some embodiments that are broken down by the body of an otherwise healthy person on average in no more than about 20 weeks, no more than about 10 weeks, or no more than about 6 weeks. Semi-absorbable examples of material include, for example, xenomaterials (Bovine Pericardial Strips and Bovine Collagen Strips; SHELHIGH NO-REACT® VACUPATCH, commercially available from Shelhigh, Milburn, N.J., USA) and the like. A commonly used non-absorbable material is expanded polytetrafluoroethylene (ePTFE commercially available from W.L. Gore, Elkton, Md., USA). A commonly used absorbable material is poly (L-lactic acid-co-epsilon-caprolactone) (Bioabsorbable SEAMGUARD®, commercially available from W L Gore and Associates Flagstaff Ariz.). A commonly used Bioremodelable material is BIODESIGN®, commercially available from Cook Inc., Bloomington Ind., and bioremoldable materials are described further below. As used herein, non-absorbable materials are materials that are not broken down by the body of an otherwise healthy person on average in greater than about 6 months, greater than about 1 year, or greater than about 5 years. A person of ordinary skill in the art will recognize that additional ranges are contemplated and are within the present disclosure. People of ordinary skill in the art will immediately appreciate that all values and ranges within the expressly stated ranges are contemplated, and are within the present disclosure.

The device may be made of any biocompatible material suitable for implantation into a mammalian body or combinations thereof. The graft may be made of a single, non-allergenic biological or synthetic material and/or a remodelable material or a combination of materials. Different portions of the device may or may not be made from different materials or combinations of materials. In general, the material selection can influence packaging and distribution of the product, with suitable packaging generally being equivalent to packaging for medical application of the material.

Suitable biological materials may be rendered non-cellular during processing to avoid immunological rejection. Such biological tissues may be implanted in potentially infected surgical fields and resist infection, unlike some synthetic preparations that may elicit a foreign body reaction or act as a nidus for infection. Suitable biological materials that may be used include, but are not limited to, heterograft material (i.e., cross-species material, such as tissue material from a non-human donor to a human recipient), allograft material (i.e., tissue material from a human cadaveric donor), and/or autograft material (i.e., where the donor and the recipient are the same individual). The material may promote angiogenesis and/or site-specific tissue remodeling. Further, any exogenous bioactive substances incorporated into an extracellular matrix (ECM) material may be from the same species of animal from which the ECM material was derived (e.g., autologous or allogenic relative to the ECM material) or may be from a different species from the ECM material source (xenogenic relative to the ECM material). In certain embodiments, ECM material can be xenogenic relative to the patient receiving the graft, and any added exogenous material(s) will be from the same species (e.g., autologous or allogenic) as the patient receiving the graft. Illustratively, human patients may be treated with xenogenic ECM materials (e.g., porcine-, bovine- or ovine-derived) that have been modified with exogenous human material(s) as described herein, those exogenous materials being naturally derived and/or recombinantly produced.

Autograft tissue can be grown from a skin biopsy of the patient. Once the fibroblasts have regenerated and formed enough new tissue, the new tissue may be injected back into the surgical site of the same patient. This process generally takes several weeks to complete, but avoids tissue rejection and disease transmission. One such product is ISOLAGEN® (available from Isolagen Inc.-Houston, Tex.).

Suitable cadaveric materials include, but are not limited to, cadaveric fascia and cadaveric dura mater. Specific suitable cadaveric allografts include, but are not limited to, ALLODERM® (LifeCell Corp.-Branchburg, N.J.), CYMETRA®, (LifeCell Corp.-Branchburg, N.J.), DERMALOGA, FASCION (Fascia Biosystems, LLC-Beverly Hills, Calif.), and SUSPEND (Mentor-Irving, Tex.). These products can be freeze-dried, or lyophilized, acellular dermal tissue from cadaveric donors. Some require reconstitution before implantation. Although disease transmission or antigenic reaction is possible, the risk may be reduced by an extensive screening and processing of the material.

Heterograft materials are taken from a donor of one species and grafted into a recipient of another species. Examples of such materials include, but are not limited to, SURGISIS® (Cook Surgical-Bloomington, Ind.), PERMACOL™ (TSL-Covington, Ga.), PELVICOL® (Bard Inc.-Murray Hill, N.J.) and PERI-GUARD™, (Bio-Vascular Inc.-St Paul, Minn.).

The materials used to form the grafts should generally be biocompatible, and in desirable embodiments, are comprised of a remodelable material. The material may have a collagenous tissue frame that remains intact to allow for ingrowth of host cells and eventual reconstruction of the host tissue itself. Remodelable collagenous materials may be provided, for example, by collagenous materials isolated from a warm-blooded vertebrate, and including a mammal. Such isolated collagenous material may be processed so as to have remodelable, angiogenic properties and promote cellular invasion and ingrowth. Remodelable materials may be used in this context to promote cellular growth on, around, and/or within tissue in or on which the device is implanted, e.g., around tissue defining an anastomosis.

Suitable remodelable materials may be provided by collagenous extracellular matrix (ECM) materials possessing biotropic properties. For example, suitable collagenous materials include ECM materials such as submucosa, renal capsule membrane, dermal collagen, dura mater, pericardium, serosa, peritoneum or basement membrane layers, including liver basement membrane. Suitable submucosa materials for these purposes include, for instance, intestinal submucosa including small intestinal submucosa, stomach submucosa, urinary bladder submucosa, and uterine submucosa. Submucosa may be obtained by harvesting such tissue sources and delaminating the submucosa from smooth muscle layers, mucosal layers, and/or other layers occurring in the tissue source. For additional information as to submucosa, and its isolation and treatment, reference can be made, for example, to U.S. Pat. Nos. 4,902,508, 5,554,389, 5,993,844, 6,206,931, and 6,099,567, which are hereby incorporated by reference herein in their entirety to the extent they do not contradict what is explicitly disclosed herein.

Submucosa or other ECM tissue may be highly purified, for example, as described in U.S. Pat. No. 6,206,931 to Cook et al., which is hereby incorporated by reference herein in its entirety to the extent it does not contradict what is explicitly disclosed herein. In some embodiments, ECM material may exhibit an endotoxin level of less than about 12 endotoxin units (EU) per gram, in further embodiments, less than about 5 EU per gram, or less than about 1 EU per gram. As additional preferences, the submucosa or other ECM material may have a bioburden of less than about 1 colony forming units (CFU) per gram or less than about 0.5 CFU per gram. Fungus levels are desirably similarly low, for example less than about 1 CFU per gram or less than about 0.5 CFU per gram. Nucleic acid levels may be less than about 5 .mu.g/mg or less than about 2 .mu.g/mg, and virus levels may be less than about 50 plaque forming units (PFU) per gram or less than about 5 PFU per gram. These and additional properties of submucosa or other ECM tissue taught in U.S. Pat. No. 6,206,931, may be characteristic of any ECM tissue used. A person of ordinary skill in the art will recognize that additional ranges are contemplated and are within the present disclosure. Persons of ordinary skill in the art will immediately appreciate that all values and ranges within the expressly stated ranges are contemplated.

A typical layer thickness for an as-isolated submucosa or other ECM tissue layer ranges from about 50 to about 250 microns when fully hydrated, and more typically from about 50 to about 200 microns when fully hydrated, although isolated layers having other thicknesses may also be obtained and used. These layer thicknesses may vary with the type and age of the animal used as the tissue source. These layer thicknesses may also vary with the source of the tissue obtained from the animal source. A person of ordinary skill in the art will recognize that additional ranges are contemplated and are within the present disclosure. Persons of ordinary skill in the art will immediately appreciate that all values and ranges within the expressly stated ranges are contemplated.

In some embodiments, the devices can comprise one or more bioactive agents. As used herein, the phrase “bioactive agent” refers to any pharmaceutically active agent that produces an intended therapeutic effect on the body to treat or prevent conditions or diseases. Such bioactive agents may be incorporated into the device material(s), coated onto the device material(s), or included in the device (or portions thereof) in any other suitable manner. For example, a bioactive agent (or a bioactive agent combined with another biocompatible material) may be coated on a device body or contained in passages formed in a device body, and be configured to release over a certain period of time.

Suitable bioactive agents may include, for example, one or more bioactive agents native to the source of the ECM tissue material. For example, a submucosa or other remodelable ECM tissue material may retain one or more growth factors including, for example, basic fibroblast growth factor (FGF-2), transforming growth factor beta (TGF-beta), epidermal growth factor (EGF), cartilage derived growth factor (CDGF), and/or platelet derived growth factor (PDGF). In addition, submucosa or other ECM materials may retain other native bioactive agents, including, for example, proteins, glycoproteins, proteoglycans, and glycosaminoglycans. For example, ECM materials may include heparin, heparin sulfate, hyaluronic acid, fibronectin, cytokines, and the like. Thus, generally speaking, a submucosa or other ECM material may retain one or more bioactive components that induce, directly or indirectly, a cellular response such as a change in cell morphology, proliferation, growth, protein or gene expression.

In addition or as an alternative to the inclusion of such native bioactive components, non-native bioactive components such as those synthetically produced by recombinant technology or other methods (e.g., genetic material such as DNA), may be incorporated into an ECM material. These non-native bioactive components may be naturally-derived or recombinantly produced proteins that correspond to those natively occurring in an ECM tissue, but perhaps of a different species. These non-native bioactive components may also be drug substances. Illustrative drug substances that may be added to material layers include, for example, anti-clotting agents, e.g., heparin, antibiotics, anti-inflammatory agents, and anti-proliferative agents, e.g., taxol derivatives such as paclitaxel. Such non-native bioactive components may be incorporated into and/or onto ECM material in any suitable manner, such as by surface treatment (e.g., spraying) and/or impregnation (e.g., soaking).

Other suitable bioactive agents include, for example: antithrombotics, including anticoagulants, antiplatelets, and fibrinolytics. Suitable anticoagulants include thrombin, Factor Xa, Factor VIIa, tissue factor inhibitors, heparin, low molecular weight heparin, covalent heparin, synthetic heparin salts, coumadin, bivalirudin (hirulog), hirudin, argatroban, ximelagatran, dabigatran, dabigatran etexilate, D-phenalanyl-L-poly-L-arginyl, chloromethy ketone, dalteparin, enoxaparin, nadroparin, danaparoid, vapiprost, dextran, dipyridamole, omega-3 fatty acids, vitronectin receptor antagonists, DX-9065a, CI-1083, JTV-803, razaxaban, BAY 59-7939, and LY-51,7717. Suitable antiplatelets include glycoprotein IIb/IIIa, thromboxane A2, ADP-induced glycoprotein IIb/IIIa, phosphodiesterase inhibitors, eftibatide, tirofiban, orbofiban, lotrafiban, abciximab, aspirin, ticlopidine, clopidogrel, cilostazol, dipyradimole, nitric oxide sources such as sodium nitroprussiate, nitroglycerin, S-nitroso and N-nitroso compounds. Suitable fibrinolytics include plasminogen activators, thrombin activatable fibrinolysis inhibitor (TAFI) inhibitors, other enzymes which cleave fibrin, alfimeprase, alteplase, anistreplase, reteplase, lanoteplase, monteplase, tenecteplase, urokinase, streptokinase, or phospholipid encapsulated microbubbles; and other bioactive materials such as endothelial progenitor cells or endothelial cells.

Other examples of suitable bioactive agents include, but are not limited to: antiproliferative/antimitotic agents including natural products such as vinca alkaloids (i.e., vinblastine, vincristine, and vinorelbine), paclitaxel, epidipodophyllotoxins (i.e., etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as (GP) II/III.sub.a inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine cladribine}); platinum coordination complexes (cisplatin, carboplatin), procarbazine, hydroxyurea, mitotane, aminoglutethimide; hormones (i.e., estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6.alpha.-methylprednisolone, triamcinolone, betamethasone, and dexamethasone), non-steroidal agents (salicylic acid derivatives i.e., aspirin; para-aminophenol derivatives i.e., acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives (cyclosporine, tacrolimus (FK-506), sirolimus (rapamycin), tacrolimus, everolimus, azathioprine, mycophenolate mofetil); angiogenic agents: vascular endothelial growth factor (VEGF), fibroblast growth factor (FGF); angiotensin receptor blockers; nitric oxide and nitric oxide donors; anti-sense oligionucleotides and combinations thereof; cell cycle inhibitors, mTOR inhibitors, and growth factor receptor signal transduction kinase inhibitors; retenoids; cyclin/CDK inhibitors; endothelial progenitor cells (EPC); angiopeptin; pimecrolimus; angiopeptin; HMG co-enzyme reductase inhibitors (statins); metalloproteinase inhibitors (batimastat); protease inhibitors; antibodies, such as EPC cell marker targets, CD34, CD133, and AC 133/CD133; Liposomal Biphosphate Compounds (BPs), Chlodronate, Alendronate, Oxygen Free Radical scavengers such as Tempamine and PEA/NO preserver compounds, and an inhibitor of matrix metalloproteinases, MMPI, such as Batimastat.

ECM materials may be free of additional, non-native crosslinking, or may contain additional crosslinking. Such additional crosslinking may be achieved by photo-crosslinking techniques, by chemical crosslinkers, or by protein crosslinking induced by dehydration or other means. However, because certain crosslinking techniques, certain crosslinking agents, and/or certain degrees of crosslinking can destroy the remodelable properties of a remodelable material, where preservation of remodelable properties is desired, any crosslinking of the remodelable ECM material may be performed to an extent or in a fashion that allows the material to retain at least a portion of its remodelable properties. Chemical crosslinkers that may be used include for example aldehydes such as glutaraldehydes, diimides such as carbodiimides, e.g., 1-ethyl-3-(3-dimethylaminopropyl)carbodiimide hydrochloride, ribose or other sugars, acyl-azide, sulfo-N-hydroxysuccinamide, or polyepoxide compounds, including for example polyglycidyl ethers such as ethyleneglycol diglycidyl ether, available under the trade name DENACOL EX810 from Nagese Chemical Co., Osaka, Japan, and glycerol polyglycerol ether available under the trade name DENACOL EX 313 also from Nagese Chemical Co. Typically, when used, polyglycerol ethers or other polyepoxide compounds will have from about 2 to about 10 epoxide groups per molecule. A person of ordinary skill in the art will recognize that additional ranges are contemplated and are within the present disclosure. Persons of ordinary skill in the art will immediately appreciate that all values and ranges within the expressly stated ranges are contemplated.

In some embodiments the materials incorporated may be sterilized prior to use. Appropriate sterilization techniques can be adapted based on recommended techniques for the materials used in the device.

In some embodiments a device or components thereof are provided that include a multilaminate material. Such multilaminate materials may include a plurality of ECM material layers bonded together, a plurality of non-ECM materials bonded together, or a combination of one or more ECM material layers and one or more non-ECM material layers bonded together. To form a multilaminate ECM material, for example, two or more ECM segments can be stacked, or one ECM segment can be folded over itself at least one time, and then the layers may be fused or bonded together using a bonding technique, such as chemical cross-linking or vacuum pressing during dehydrating conditions. An adhesive, glue or other bonding agent may also be used in achieving a bond between material layers. Suitable bonding agents may include, for example, collagen gels or pastes, gelatin, or other agents including reactive monomers or polymers, for example cyanoacrylate adhesives. Surgical adhesives are commercially available. In addition, bonding can be achieved or facilitated between ECM material layers using chemical cross-linking agents such as those described above. A combination of one or more of these, optionally with dehydration-induced bonding, may also be used to bond ECM material layers to one another.

A variety of dehydration-induced bonding methods may be used to fuse together portions of an ECM material. In one embodiment, multiple layers of ECM material are compressed under dehydrating conditions. In this context, the term “dehydrating conditions” includes any mechanical or environmental condition which promotes or induces the removal of water from the ECM material. To promote dehydration of the compressed ECM material, at least one of the two surfaces compressing the matrix structure may be water permeable. Dehydration of the ECM material can optionally be further enhanced by applying blotting material, heating the matrix structure or blowing air, or other inert gas, across the exterior of the compressed surfaces. One particularly useful method of dehydration bonding ECM materials comprises lyophilization.

Another method of dehydration bonding comprises pulling a vacuum on the assembly while simultaneously pressing the assembly together. This method can be referred to as vacuum pressing. During vacuum pressing, dehydration of the ECM materials in forced contact with one another effectively bonds the materials to one another, even in the absence of other agents for achieving a bond, although such agents can be used while also taking advantage at least in part of the dehydration-induced bonding. With sufficient compression and dehydration, the ECM materials form a generally unitary ECM structure.

In some embodiments, drying and other operations are performed under relatively mild temperature exposure conditions that minimize deleterious effects upon any ECM materials being used, for example native collagen structures and potentially bioactive substances present. Thus, drying operations conducted with no or substantially no duration of exposure to temperatures above human body temperature or slightly higher (e.g., no higher than about 38° C.) may be performed in some embodiments. These include, for example, vacuum pressing operations at less than about 38° C., forced air drying at less than about 38° C., or either of these processes with no active heating—at about room temperature (i.e., about 25° C.) or with cooling. Relatively low temperature conditions also, of course, can include lyophilization conditions. A person of ordinary skill in the art will recognize that additional ranges are contemplated and are within the present disclosure. People of ordinary skill in the art will immediately appreciate that all values and ranges within the expressly stated ranges are contemplated, and are within the present disclosure.

The device may comprise biocompatible materials derived from a number of biological polymers, which can be naturally occurring or the product of in vitro fermentation, recombinant genetic engineering, and the like. Purified biological polymers can be appropriately formed into a substrate by techniques such as weaving, knitting, casting, molding, and extrusion. Suitable biological polymers include, without limitation, collagen, elastin, keratin, gelatin, polyamino acids, polysaccharides (e.g., cellulose and starch) and copolymers thereof.

Suitable biocompatible materials may also include, for example, a variety of synthetic polymeric materials including bioresorbable and/or non-bioresorbable plastics. Bioresorbable, or bioabsorbable polymers that may be used include, for example, poly(L-lactic acid), polycaprolactone, poly(lactide-co-glycolide), poly(hydroxybutyrate), poly(hydroxybutyrate-co-valerate), polygalactin, hyaluronic acid, polydioxanone, polyorthoester, polyanhydride, poly(glycolic acid), poly(D,L-lactic acid), poly(glycolic acid-co-trimethylene carbonate), polyhydroxyalkanaates, polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, and polyphosphazenes. These or other bioresorbable materials may be used, for example, where only a temporary blocking or closure function is desired, and/or in combination with non-bioresorbable materials where only a temporary participation by the bioresorable material is desired.

Non-bioresorbable, or biostable polymers that may be used include, for example, polytetrafluoroethylene (PTFE) (including expanded PTFE), polyethylene terephthalate (PET), polyurethanes, silicones, and polyesters and other polymers such as polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins, polycarbonates; polyoxymethylenes; polyimides; polyethers; epoxy resins, polyurethanes; rayon; and rayon-triacetate.

The biological or synthetic material may assist in reconstruction of the host tissues, elicit little immunological reaction, and have some inherent resistance to infection. Such material may allow incorporation of the device into the host tissue of the recipient (rather than complete absorption of the device into the surrounding tissue), thereby reinforcing the anastomosis.

In some embodiments, a drug, such as an antibiotic, is incorporated into the device, as an extra precaution or means of treating any residual around the anastomosis. The device may also be used in conjunction with a sealant or sclerosing solution which may be used to seal or glue the device to the bowel wall. Several possible sealants or glues are commercially available. One of the more commonly used sealants is fibrin glue, commercially available as TISSEAL (Baxter Inc.). The glue is prepared by mixing coagulation activation factors with fibrinogen, which react to form fibrin. The fibrin forms a matrix, which acts as a scaffold for tissue ingrowth and results in the adherence of the device to the anastomosis.

### Device in Situ

FIGS. 9 and 13-16 illustrate a possible final location of the proximal and distal sleeves. The proximal and distal sleeves are held in place by the fixation to the actual staple line buttress at the anastomosis. The proximal sleeve is located outside the colon which makes the device easier to apply intraoperatively. In addition, by avoiding an intraluminal location of the device, this prevents prolapse of the device through the anastomosis and prevents potential bowel obstruction from the device itself. The distal aspect of the device is located within the lumen of the rectum, and prevents anastomotic leak from the luminal aspect of the bowel.

In a further embodiment of the device, the proximal or distal bowel or both may be sutured or otherwise anastomosed to the device at a site separate from the anastomosis to further reduce tension at the anastomosis. For example, suturing the proximal aspect of the proximal sleeve to the proximal bowel may reduce tension at the anastomosis itself so further reducing the risk of anastomotic leak.

In a further embodiment of the device, a lateral defect extending all or part of the way along one side of the side of the cup or sleeve allows for insertion of the colon mesentery and avoids ischemia at the anastomosis

In summary, the device incorporates a sleeve to protect the proximal and distal few centimeters of bowel, in addition to reinforcing the actual anastomosis itself by means of the anastomotic buttress. Depending on the absorbability of the material, the proximal sleeve may be incorporated into the anastomosis and the distal aspect of the sleeve may slowly dissolve and be passed through the rectum days or weeks later.

The device also avoids the need for an additional incision in the colon to introduce the device. The sleeves are held in position by virtue of their attachment to the staple buttress. The device protects the proximal aspect of the anastomosis by the outer sleeve, as well as the inner sleeve which protects the distal aspect of the anastomosis, rather than just the distal aspect of the anastomosis.

The device with two cups formed from a tissue based material was testing in animal studies. Feasibility studies were performed in 4 pigs. Each pig had a L colon resection, and were euthanized 7 days later to examine the device and anastomosis. In 3 pigs the device incorporated well and the anastomosis was well healed. In the 4th pig, there was an inadvertent (and at the time of surgery, unrecognized) anastomotic defect related to the stapler malfunction. The leak was discovered in pig #4 when it was euthanized a week later. The device had successfully prevented the leak and averted spillage of stool and prevented peritonitis and death. The inadvertent stapler malfunction correspondingly supported proof of concept.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein.

What is claimed is:

1. A system for performing a medical procedure comprising:
a) a stapler comprising:
a detachable anvil head comprising an anvil surface and a hollow rod comprising a lumen protruding from the anvil surface, a stapler shaft comprising a stapler surface and a spike protruding from the stapler surface, and a shape cutter,
wherein the anvil surface comprises a groove,
wherein the stapler surface comprises a staple port and a cutter port
wherein the cutter can advance through the cutter port, and
wherein the spike is proportioned to fit inside the lumen of the hollow rod; and
b) a support structure comprising:
a first shield piece comprising a first support surface and at least one first wall comprising a first bottom edge wherein the first bottom edge is attached to the first support surface and a second shield piece comprising a second support surface,
wherein the second shield piece is not attached to the first shield piece,
wherein the first support surface and/or the second support surface comprises a hole, and
wherein the first support surface and the second support surface align with the anvil surface and the stapler surface to provide for the delivery of a staple through the first support surface and the second support surface.

2. The system of claim 1 wherein the second shield piece further comprises at least one second wall comprising a second bottom edge wherein the second bottom edge is attached to the second support surface.

3. The system of claim 2 wherein the support structure further comprises a radially extendable framework.

4. The system of claim 1 wherein the first wall further comprises a top edge and wherein the first shield piece further comprises a ring located at the top edge of the first wall.

5. The system of claim 1 wherein the first wall is about 2.5 mm to about 10 cm long.

6. The system of claim 1 wherein the support structure comprises a sheet of biocompatible polymer and/or collagenous material.

7. The system of claim 6 wherein the sheet of biocompatible polymer and/or collagenous material is absorbable in no more than about 20 weeks.

8. The system of claim 6 wherein the sheet of biocompatible polymer and/or fibrous material is a bioremodelable material.

9. The system of claim 8 wherein the bioremodelable comprises an extracellular matrix material.

10. The system of claim 1 wherein the support structure further comprises a bioactive agent.

11. A method for forming an anastomosis, the method comprising:
placing a first attachment site near the distal end of a colon segment into contact with a first shield piece comprising a first surface,
placing a second attachment site of the rectum into contact with a second shield piece comprising a second surface,
wherein the first shield piece further comprises at least one first wall comprising a first bottom edge wherein the first bottom edge is attached to the first surface and/or the second shield piece further comprises at least one second wall comprising a second bottom edge wherein the second bottom edge is attached to the second surface,
inserting a tip of a pointed attachment member through the first surface, tissue at the first attachment site, the second surface, and tissue at the second attachment site, creating an affixed section, and
cutting through the affixed section to create a lumen.

12. The method of claim 11 wherein the pointed attachment member comprises surgical staples.

13. The method of claim 11 further comprising stitching an anvil head into the colon prior to creating an affixed section.

14. The method of claim 11 wherein the first wall further comprises a first top edge and the method further comprises attaching the first top edge of the first wall to the colon proximal to the affixed section.

15. A medical apparatus for protecting an anastomosis comprising a first shield piece comprising a first surface and a second shield piece comprising a second surface, wherein the second shield piece is not attached to the first shield piece,
wherein the first shield piece further comprises at least one first wall comprising a first bottom edge wherein the first bottom edge is attached to the first surface and/or the second shield piece further comprises at least one second wall comprising a second bottom edge wherein the second bottom edge is attached to the second surface,
wherein the first surface and/or the second surface comprises a hole, and
wherein the first shield piece and the second shield piece each independently comprise a sheet of biocompatible polymer and/or collagenous material with an average thickness of no more than about 5 millimeters and are sterile.

16. The medical apparatus of claim 15 wherein the first wall further comprises a top edge and wherein the first shield piece further comprises a radially extendable ring located at the top edge of the first wall.

17. The medical apparatus of claim 15 wherein the second shield piece comprises the at least one second wall comprising the second bottom edge wherein the second bottom edge is attached to the second surface and wherein the first wall and/or the second wall is about 1 mm to about 10 cm long.

18. The medical apparatus of claim 15 wherein the sheet of biocompatible polymer and/or fibrous material is absorbable in no more than about 20 weeks.

19. The medical apparatus of claim 15 wherein the sheet of biocompatible polymer and/or fibrous material is a bioremodelable material and wherein the bioremodelable comprises an extracellular matrix material.

20. The medical apparatus of claim 15 further comprising a bioactive agent.

21. The medical apparatus of claim 15 wherein the first wall and/or the second wall further comprises a radially expandable framework.
